(19) United States
(12) Patent Application Publication
(10) Pub. No.: US 2005/0154060 A1
(43) Pub. Date: Jul. 14, 2005

(54) Anti-inflamatory inhalation pharmaceutical composition

(75) Inventors: Arthur Deboeck, Gurabo, PR (US); Francis Vanderbist, Waterloo (BE); Phillippe Baudier, Waterloo (BE)

Correspondence Address: LOWE HAUPTMAN GILMAN AND BERNER, LLP, 1700 DIAGONAL ROAD, SUITE 300 /310, ALEXANDRIA, VA, 22314, US

(73) Assignee: Galephar Pharmaceutical Research, Inc., Juncos, PR (US)

(21) Appl. No.: 11/009,017

(22) Filed: Dec. 13, 2004

Related U.S. Application Data
(60) Provisional application No. 60/528,482, filed on Dec. 11, 2003.

(51) Int. Cl. A61K 31/198

(52) U.S. Cl. 514/562

(57) ABSTRACT

The present invention is directed to the discovery of the anti-inflammatory properties of basic amino salts of acetylcysteine when administered directly to the lungs and/or the nose. This observed inflammation reduction in the lungs and/or the nasal cavity is confirmed by the diminution of at least one of the following inflammatory markers IL_6 and IL_8 and/or the increase in the lungs and/or the nasal cavity of the anti-inflammatory markers IL_10. The present invention also discloses pharmaceutical compositions for pulmonary and/or nasal administration of basic amino salts of acetylcysteine characterised that they have anti-inflammatory properties and may be useful to treat inflammatory lung and/or nasal conditions found in illnesses such as COPD, asthma, cystic fibrosis and the like.

### BACKGROUND OF THE INVENTION

N-Acetyl-L-Cysteine or Acetylcysteine is a well known medication useful as a mucolytic and for the treatment of acetaminophen intoxication when administered by oral and/or intravenous route.

Mucolytic treatment consists in acetylcysteine oral administration of daily amounts comprised between 200 mg and 800 mg divided in 2 or 3 doses.

For the treatment of acetaminophen overdoses, the therapy involves emptying the gastrointestinal tract with ipecac or by means of lavage with a large-bore tube. If drug blood levels exceed 250 μg/mL four hours after ingestion, the overdose is probably severe. Because hepatotoxicity is caused by a gluthathione-depleting metabolite of acetaminophen, the sulfhydryl donor acetylcysteine is an effective antidote. Acetylcysteine is given orally as five percent solution diluted with three volumes of a soft drink. The initial loading dose is 140 mg/kg; thereafter a dose of 70 mg/kg is given every four hours for an additional 17 doses. If the patient is unable to swallow, the solution is administered by gastric or enteral tube. Treatment is most effective when it is begun within eight hours of ingestion but can still be beneficial if it is delayed for as long as 24 hours. In one series of 2,540 poisonings, there were no deaths among persons who received the antidote within 16 hours. Intravenous acetylcysteine (300 mg/kg, given during a 20 hour period) has been used in Europe and Canada. The 72 hour oral treatment appears to be as effective as the 20 hour intravenous regimen.

In both these conditions it is believed that acetylcysteine when taken orally is, via hepatic metabolisation, a source of glutathione a natural tripeptide circulating antioxidant. Therefore mucolytic and acetaminophen detoxification activities are de factum the results of the action of glutathione, and the acetylcysteine is given as a precursor (source) of the glutathione.

U.S. Pat. No. 4,847,282 discloses water-soluble acetylcysteine salts, useful as mucolytic agents, consisting of the reaction products of acetylcysteine with at least one basic amino-acid, the later being preferably selected from the group comprising arginine, lysine, histidine and ornithine. These water soluble salts, having a neutral pH are suitable for pulmonary administration. Also the patent discloses that these new salts of acetylcysteine do not produce bronchospasms, contrary to the parent product acetylcysteine, when administered to the lungs.

EP Patent Application 0876814A1 and PCT/BE03/00048 discloses inhalation pharmaceutical preparations and devices suitable for pulmonary administration of active drugs and in particular the highly soluble basic amino salts of acetylcysteine. One example of such salts is L-Lysine-N-Acetylcysteinate, also called Nacystelyn or NAL.

Pharmaceutical compositions derived from these 2 patents teach pharmaceutical compositions useful to administer acetylcysteine or a salt thereof, directly to the lungs and/or nose. This very effective method of administration is safe since it does not produce bronchospasms.

A new clinical trial has been performed with a dry powder inhaler (DPI) formulation of NAL in moderate to severely ill COPD patients. The study was performed with two doses of NAL DPI: 20 mg twice a day and 40 mg twice a day versus placebo.

The complete title of the study was “A 12-week, multicentre, randomised, double-blind, placebo controlled, pilot study of the safety and efficacy of Nacystelyn (20 mg b.i.d. and 40 mg b.i.d.) in patients with chronic obstructive pulmonary disease (COPD).”

NAL pulmonary administration was performed using a Dry Powder Inhaler consisting of a capsule containing NAL and a carrier, and of an inhalation device.

The primary end-point of the study was to evaluate the effect of NAL, versus placebo, on the quality of life in COPD patients between baseline values (T0) and after 12 weeks of treatment (T12).

During the same study, sputum of COPD patients was collected at baseline (T0), after 4 weeks (w4) and after 12 weeks of treatment (T12).

The purpose of collecting sputum was to assess if NAL DPI administration could affect (increase or decrease) lung inflammation parameters in such patients. Lung inflammation may be reflected by the sputum concentration of some pro-inflammatory markers, called cytokines, such as interleukine 6 (IL-6) and interleukine 8 (IL-8) or anti-inflammatory markers, such as interleukine 10 (IL-10).

It is understood that, the lower the concentration of pro-inflammatory markers in the lung, the lower the lung inflammation status.

After collection of the mucus, the said mucus was centrifugated and the cytokines IL-8 and IL-6 were quantified using a validated analytical method. The results are presented in Table 1. It should be noted that only subjects presenting a quantifiable value of cytokines at the time considered were taken into account.

TABLE 1
change in inflammatory markers IL-6 and IL-8, expressed in percent
changes [%] versus baseline after administration of placebo,
20 mg and 40 mg of NAL twice a day during 4 and 12 weeks (the
more the negative change [%], the better)

| Week | Placebo | 20 mg | 40 mg |
|---|---|---|---|
| IL-6 | | | |
| 4 | 8 | 24 | 70 |
| 12 | 51 | −17 | −42 |
| IL-8 | | | |
| 4 | +7 | −13 | −2 |
| 12 | +29 | −17 | +3 |

As seen, NAL induces a decrease of IL-6 in the sputum of COPD patients in a dose related manner. After 12 weeks of treatment, the concentration in IL-6 decreased by 17% and 42% respectively after administration of NAL 20 mg or 40 mg while the levels of IL-6 increased by 51% after administration of placebo for 12 weeks. NAL is also able to significantly decrease the IL-8 levels in the sputum. However, for this marker, the maximal effect is obtained with the 20 mg dose. Indeed, after 12 weeks of treatment of NAL 20 mg and 40 mg, the levels of IL-8 decreased by 17% and increased by 3% respectively, while during the same period, the levels of IL-8 in sputum of patients taking placebo increased by 29%.

So it has surprisingly been demonstrated in the present study that administration of Nacystelyn to patients with COPD is able to decrease the level of lung inflammation in those patients.

This is extremely surprising since the product was administered directly to the lungs, the NAL cannot be converted into GSH which is recognised by the scientific community as a substance with a known anti-inflammatory substance.

1. A water soluble N-acetyl-L-cysteine salt useful for inhalation as anti-inflammatory drug characterised in that it is formed by the reaction product between N-acetyl-L-cysteine and at least one basic amino-acid.

2. A salt as claimed in claim 1 characterised in that the basic amino salts are selected from the group comprising arginine, lysine, histidine and ornithine.

3. A salt as claimed in claim 1, characterised in that the basic amino-acid is levorotary amino-acid, a dextrorotary amino-acid or a mixture of said acids.

4. As salt as claimed in claim 1, characterised in that it contains about 1 mole of basic amino-acid per mole of N-acetyl-L-cysteine.

5. A pharmaceutical composition, comprising as active product at least one water-soluble N-acetyl-L-cysteine salt according to any of claims 1 to 4, in association with at least a suitable excipient and/or at least another therapeutical agent.

6. A pharmaceutical composition for inhalation containing at least a salt of claim 1 for administration by inhalation in the treatment of an inflammatory or obstructive respiratory disease.

7. The composition or combination of claim 1, where in the composition contains in addition to the acetyl basic amino salt at least one pharmaceutically acceptable carrier.

8. The composition of claim 5 suitable for an inhalable dry powder inhaler comprising micronized particles of at least one salt of claim 1 advantageously mixed with one or more acceptable excipient(s).

9. The composition of claim 7, wherein the composition or combination is in a form suitable for inhalation through a monodose inhaler device.

10. The composition of claim 7, wherein the composition or combination is in a form suitable for inhalation through a multiple-dose inhaler.

11. The composition of claim 7, characterised that at least one containing in addition of the excipients is selected from the group of saccharides.

12. The composition of claim 10, characterised in that the saccharides is lactose.

13. The composition or combination of claim 1, wherein the inhaled salt of claim 1 is an inhalable pressurised metered dose inhaler.

14. The composition or combination on claim 12, wherein the composition contains at least a propellant wherein the salt of claim 1 are either dissolved or dispersed.

15. The composition or combination of claim 13, wherein the propellant is chlorofluorocarbon derivative.

16. The composition or combination of claim 13, where the propellant is a hydrofluorocarbon derivative.

17. The composition of claim 1, wherein the composition is an inhalable nebulizable composition comprising a solution and/or a dispersion of such salts in ana aqueous medium.

18. The composition or combination of claim 1, which is a dry powder inhaler comprising a capsule containing from 1 to 50 mg of L-lysine N-acetylcysteineate.

19. The composition or combination of claim 17, which is a dry powder inhaler in a capsule containing from 2 to 20 mg of L-lysine N-acetylcysteinate.
